(19) United States
(12) Patent Application Publication
(10) Pub. No.: US 2018/0221468 A1
(43) Pub. Date: Aug. 9, 2018

(54) HAEMOPHILUS INFLUENZAE IMMUNOGEN

(71) Applicant: Iowa State University Research Foundation, Inc., Ames, IA (US)

(72) Inventors: David Verhoeven, Ames, IA (US); Brian Lee, Ames, IA (US)

(21) Appl. No.: 15/874,659

(22) Filed: Jan. 18, 2018

Related U.S. Application Data
(60) Provisional application No. 62/448,026, filed on Jan. 19, 2017.

(51) Int. Cl. A61K 39/102; A61K 9/51; C12N 9/02; G01N 33/68

(52) U.S. Cl. CPC A61K 39/102; A61K 9/5169; A61K 9/5115; A61K 2039/575; C12Y 102/04001; G01N 33/6854; C12N 9/0008

(57) ABSTRACT

The present invention provides immunogens which can be used for vaccines against non-typeable Haemophilus influenzae (NTHi). The protein targeted is NTHi PDE1 and is highly conserved making it a vital component necessary for bacterial pathogenesis. The protein preferably includes the C terminal fragment after cleavage between Asn269 and Gly270, when compared to the wild-type PDE1 amino acid sequence. Nucleic acid sequences, amino acid sequences, immunogenic compositions, treatments and methods of diagnosis are disclosed.

### CROSS-REFERENCE TO RELATED APPLICATIONS

This application claims priority to provisional application U.S. Ser. No. 62/448,026, filed Jan. 19, 2017, which is incorporated herein by reference in its entirety.

### FIELD OF THE INVENTION

The present invention is directed to methods and compositions for the treatment and/or prevention of non-typeable Haemophilus influenzae (NTHi) infection and/or disease caused by H. influenza, such as otitis media.

### BACKGROUND OF THE INVENTION

Non-typeable Haemophilus influnzae (NTHi) is a significant respiratory pathogen that contributes substantially to millions of middle ear infections in young children yearly, and also the many cases of pneumonia in the elderly. The pathogen is also one for which no current efficacious vaccine exists. Moreover, despite many NTHi vaccines tested in mice, none have proven highly protective in human clinical trials. Thus a significant gap in the field exists with respect to the ideal immunogens for use as a vaccine.

In screening serum samples from otitis prone and non-prone children against NTHi proteins, recent work by the Inventors has shown that the former do not mount any antibody responses to pyruvate dehydrogenase E1 component (PDE1) a key protein involved in the Krebs/TCA cycle. In contrast, every non-prone child tested had antibodies that reacted to PDE1. In addition to this finding, unexpectedly, recent work by the Inventors has also shown that NTHi may express a truncated version of PDE1, which may serve as an adherence/virulence factor. This further supports the potential for use of PDE1, or truncated versions thereof, for use as vaccine immunogens, especially given how and why NTHi may be using PDE1 for activities outside of metabolism. As demonstrated by studies of surface displayed glycolytic enzymes in Mycoplasma pneumoniae, there is precedence for PDE1 to moonlight as an adherence factor capable of binding to extracellular matrix proteins (fibronectin and plasminogen) and contribute to pathogenesis.

Ninety-four percent of all acute otitis media (AOM) or pneumonia occurs in the context of a preceding viral infection, the environmental or immune changes caused by a respiratory virus are significant in allowing NTHi to leave the nasal mucosal and cause invasive diseases. For the most part, NTHi nasal colonization is transient with no disease evident. A preceding viral infection damages epithelial cells promoting bacterial adherence. The immune response increases mucus secretion that allows bacteria to efflux into the Eustachian tubes and become resident in the middle ears, usually within 3-5 days of viral infection. The innate immune response usually limits nutrient availability during a viral infection, but knowledge is limited about the correlation between nutrient availability and changes in NTHi that lead to invasive pathogenesis. As seen in M. pneumoniae, surface-displayed PDE1 may bind to fibronectin or plasminogen, which are both expressed in the middle ear and lungs. However, PDE1 from M. pneumoniae consists of two subunits with α₂β₂ topology, but in NTHi, the α and β subunit functions are combined into one polypeptide chain to form a PDE1 homodimer.

Previous work has not examined this aspect of NTHi, nor has previous work identified PDE1, or truncated versions of PDE1, as a potential immunogen.

### PDE1

The pyruvate dehydrogenase complex (PDC) comprises three enzymes that catalyze the oxidative decarboxylation of pyruvate and transfer of an acetyl group to coenzyme A (CoA). PDE1 utilizes a Mg²⁺ bound thiamine pyrophosphate (TPP) cofactor for the oxidative decarboxylation of pyruvate and reductive acetylation of a lipoamide moiety attached to the dihydrolipoyl transacetylase E2 component (PDE2). PDE2 catalyzes the transfer of the acetyl group to form acetyl-CoA leaving a fully reduced dihydrolipoamide moiety. Finally, dihydrolipoyl dehydrogenase E3 component (PDE3) oxidizes the dihydrolipoamide moiety and releases NADH. NTHi is dependent on host derived NAD for the PDE3 cofactor, which is potentially acquired from human catalase. For PDE1, the interaction with TPP is required for activity and may be required to maintain structural integrity. Dissociation of TPP may cause PDE1 to self-cleave and capable of being transported to the periplasmic space. There are two TPP binding sites in the PDE1 dimer at the N-terminal domain (191-264) and C terminal domain (520-640) and thus any cleavage likely removes the N-terminal domain and causes dissociation of the PDE1 dimer. The monomeric form of PDE1 is presumably more susceptible to self-cleavage.

Of interest, NTHi and H. influenzae are missing most of the enzymes of the oxidative branch of the Krebs cycle. Thus under aerobic conditions, the acetyl-CoA produced by the pyruvate dehydrogenase complex (PDC) is used to generate ATP from acetyl-P. Under anaerobic conditions, NTHi does require PDE1 for pyruvate metabolism and instead utilizes pyruvate formate lyase (PFL) to produce acetyl-CoA without generating NADH. It is believed this could allow PDE1 to perform other cellular functions in NTHi; but surprisingly, there are very few studies on this enzyme's function in these bacteria.

PDE1, along with the other components of the PDC, associates in some way with an unknown surface protein in E. coli and, through binding of CXCL 10, causes bactericidal activity. However, the role of PDE1 in NTHi, as previously stated, has not been described and may have an important role in bacterial pathogenesis outside of energy metabolism. PDE1 might self-cleave and migrate to the periplasm where its function is presently unknown. This cleavage could be occurring as a signal to switch from aerobic metabolism to anaerobic metabolism, serve as an electron shuttle in the periplasm, be caused by Mg²⁺ chelation during the innate immune release of extracellular ATP, or by limited nutrient availability.

Understanding the role of pyruvate dehydrogenase E1 in NTHi is important given the absence of the complete Krebs/TCA cycle. In particular, the determination of whether PDE1 truncates and imparts another function onto the protein; where and how truncated PDE1 localizes to exert alternative function; and whether truncated PDE1 plays a significant role in NTHi pathogenesis in the mucosa all have important implications for the effective treatment and prevention of NTHi infection.

### BRIEF SUMMARY OF THE INVENTION

The present invention provides a new significant immunogen which can be used for vaccines against NTHi. The protein targeted by the immunogen is highly conserved and thus targeting it could block out a vital component necessary for bacterial pathogenesis.

In one embodiment, provides novel immunogens comprising the polypeptide NTHi PDE1 or a polypeptide at least 50% identical, at least 60% identical, at least 70% identical, or at least 80% identical thereto. In a preferred embodiment, the NTHi polypeptide is the C-terminal fragment after cleavage between Asn269 and Gly270, when compared to the wild-type PDE1 amino acid sequence.

In one embodiment, provides a protein nanoparticle comprising the immunogens of the present invention.

In one embodiment, provides isolated nucleic acid molecules encoding the immunogenic polypeptides disclosed herein.

In one embodiment, the isolated nucleic acid molecule is operably linked to a promoter.

In one embodiment, provides a vector comprising the nucleic acid encoding the immunogenic polypeptides of the invention.

In another embodiment, administration of an effective amount of the immunogen, protein nanoparticle, nucleic acid or vector induces a neutralizing response to Non-typeable Haemophilus influenzae in a subject.

In another embodiment, provides a method for generating an immune response to Non-typeable Haemophilus influenzae in a subject in need thereof.

In another embodiment, provides a method for preventing or treating a Non-typeable Haemophilus influenzae infection in a subject.

In another embodiment, provides a kit comprising the immunogens of the present invention.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A shows that the truncated PDE1 from NTHi reveals a potential transport signal peptide with resemblance to the GG-motif in colicin V. Based on the structure of PDE1 from E. coli, the self-cleavage site in H. influenzae after Asn269 lies between the thiamine pyrophosphate (TPP) binding site and the glycine zipper dimer interface. Sequence alignments reveal that PDE1 is highly conserved amongst Gram-negative bacteria (SEQ ID NOs: 3-12) (Waterhouse et al., Bioinformatics, 25:1189-91, 2009, and Edgar, Nucleic Acids Res. 32:1792-7, 2004). The sequence of H. influenzae is unique in having asparagine at position 269, which has the potential for self-cleavage through a succinimide ring intermediate (T. E. Creighton, Proteins, Macmillan, 1993). FIG. 1B shows PDE1 from E. coli as a homodimer bound to two TPP cofactors with one subunit in gray and the other colored in a rainbow schema from blue at the N-terminus to red at the C-terminus (Arjunan et al., Biochemistry, 41:5213-21, 2002). The potential cleavage site after Asn269 in H. influenzae is indicated. FIG. 1C shows that after cleavage of the N-terminal region, the TPP binding site is destroyed and the dimer interface is disrupted. The appearance as a surface antigen suggests that PDE1 is transported through the bacterial inner membrane after self-cleavage.

FIG. 2 shows only AOM non-prone children mount an antibody response to PDE1. Serum from 25 non-prone and 25 prone children (aged 9-24 months with a history of NTHi infections) was reacted, and none of the prone children were found to have mounted an antibody response. Representative western blot is shown. The protein band was confirmed to be PDE1 by sequencing.

FIG. 3A shows PDE1 is cleaved in NTHi to yield 69 kD and 30 kD products. NTHi was grown in BHI media with Heme and NAD in log phase or stationary phase. Additional bacteria was grown on chocolate agar plates overnight. Not shown is E coli control which did not have banding for PDE1 at these two sizes. FIG. 3B shows western blotting of aseE gene (PDE1) with detection by antibody to 6×His tag as expressed in E. coli. FIG. 3C shows coomassie staining of induced (two left lanes) and uninduced (two right) showing expression of this protein in E. coli.

FIGS. 4A-B show PDE1 is in the periplasm. NTHi was grown under permissive culture conditions, and the surface membrane disrupted by brief incubation in low salt buffer. PDE1 antibodies were then used to probe for PDE1 expression. FIG. 4A is a negative control and FIG. 4B shows staining of the periplasm of log growth NTHi.

### DETAILED DESCRIPTION OF THE INVENTION

The following definitions and introductory matters are provided to facilitate an understanding of the present invention.

Numeric ranges recited within the specification, including ranges of “greater than,” “at least,” or “less than” a numeric value, are inclusive of the numbers defining the range and include each integer within the defined range.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes.

As used herein, “adjuvant” means a vehicle used to enhance antigenicity. In some embodiments, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, and TNF-α, IFN¬y, G-CSF, LFA-3, CD72, B7-1, B7-2, OX-40 L, 4-1BBL and toll-like receptor (TLR) agonists, such as TLR-9 agonists. In some embodiments, the ADJUPLEX™ (Advanced BioAdjuvants) can be used with any of the immunogens of the present invention to elicit an immune response. The person of ordinary skill in the art is familiar with adjuvants (see, e.g., Singh (ed.) Vaccine Adjuvants and Delivery Systems. Wiley-Interscience, 2007). Adjuvants can be used in combination with the disclosed immunogens.

As used herein, the term “administration” refers to the introduction of a composition into a subject by a chosen route. Administration can be local or systemic. For example, if the chosen route is intravenous, the composition (such as a composition including a disclosed immunogen) is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

The term “agent” means any substance or any combination of substances that is useful for achieving an end or result; for example, a substance or combination of substances useful for inhibiting NTHi infection in a subject. Agents include proteins, nucleic acid molecules, compounds, small molecules, organic compounds, inorganic compounds, or other molecules of interest. An agent can include a therapeutic agent (such as an anti-bacterial agent), a diagnostic agent or a pharmaceutical agent. In some embodiments, the agent is a protein agent (such as an NTHi PDE1 polypeptide or immunogenic fragment thereof), or an anti-bacterial agent. The skilled artisan will understand that particular agents may be useful to achieve more than one result.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

The term “conservatively modified variants” applies to both amino acid and nucleic acid sequences. With respect to particular nucleic acid sequences, a conservatively modified variant refers to those nucleic acids, which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid, which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, generation of immune response, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids, which are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W.H. Freeman and Company. Further, the term “amino acid substitutions” means the replacement of one amino acid in a polypeptide with a different amino acid or with no amino acid (i.e., a deletion). In some examples, an amino acid in a polypeptide is substituted with an amino acid from a homologous polypeptide, for example, and amino acid in a recombinant or synthesized Haemophilus influenza PDE1 polypeptide can be substituted with the corresponding amino acid from a Haemophilus spp. PDE1 polypeptide.

As used herein, “antibody” means an immunoglobulin, antigen-binding fragment, or derivative thereof, which specifically binds and recognizes an analyte (antigen) such as PDE1, an antigenic fragment thereof, or a dimer or multimer of the antigen. The term “antibody” is used herein in the broadest sense and encompasses various antibody structures, including but not limited to monoclonal antibodies, polyclonal antibodies, multi-specific antibodies (e.g., bi-specific antibodies), and antibody fragments, so long as they exhibit the desired antigen-binding activity.

Non-limiting examples of antibodies include, for example, intact immunoglobulins and variants and fragments thereof known in the art that retain binding affinity for the antigen. Examples of antibody fragments include but are not limited to Fv, Fab, Fab′, Fab′-SH, F(ab′)2; diabodies; linear antibodies; single-chain antibody molecules (e.g. scFv); and multi-specific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2nd Ed., Springer Press, 2010).

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space. The CDRs are primarily responsible for binding to an epitope of an antigen.

A “monoclonal antibody” is an antibody produced by a single clone of B-lymphocytes or by a cell into which nucleic acid encoding the light and heavy chains of a single antibody have been transfected, or a progeny thereof. Monoclonal antibodies are produced by methods known to those of skill in the art, for instance by making hybrid antibody-forming cells from a fusion of myeloma cells with immune spleen cells. These fused cells and their progeny are termed “hybridomas.” In some examples monoclonal antibodies are isolated from a subject. Monoclonal antibodies can have conservative amino acid substitutions which have substantially no effect on antigen binding or other immunoglobulin functions. (See, for example, Harlow & Lane, Antibodies, A Laboratory Manual, 2^{nd} ed. Cold Spring Harbor Publications, New York (2013).)

As used herein, the term “antigen” refers to a compound, composition or substance that can stimulate the production of antibodies or a T cell response in an animal, including compositions that are injected or absorbed into an animal. An antigen reacts with the products of specific humoral or cellular immunity, including those induced by heterologous or synthesized antigens, such as the disclosed NTHi antigens. Examples of antigens include, but are not limited to, polypeptides, peptides, lipids, polysaccharides, combinations thereof (such as glycopeptides) and nucleic acids containing antigenic determinants, such as those recognized by an immune cell. In some examples, antigens include peptides derived from a pathogen of interest, such as NTHi. An antigen can include one or more epitopes.

Conditions which allow an antibody or antigen binding fragment thereof to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Harlow & Lane, Antibodies, A Laboratory Manual, 2^{nd} ed. Cold Spring Harbor Publications, New York (2013) for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists a temperature above 0° C. and below 50° C. Osmolarity is within the range that is supportive of cell viability and proliferation.

As used herein, “contacting” refers to the placement in direct physical association; includes both in solid and liquid form, which can take place either in vivo or in vitro. Contacting includes contact between one molecule and another molecule, for example the amino acid on the surface of one polypeptide, such as a peptide, that contacts another polypeptide. Contacting can also include contacting a cell for example by placing a polypeptide in direct physical association with a cell.

The term “control” refers to a reference standard. In some embodiments, the control is a negative control sample obtained from a healthy patient. In other embodiments, the control is a positive control sample obtained from a patient diagnosed with NTHi infection. In still other embodiments, the control is a historical control or standard reference.

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

As used herein, “detecting” refers to identifying the existence, presence, or fact of something. General methods of detecting are known to the skilled artisan and may be supplemented with the protocols and reagents disclosed herein. For example, included herein are methods of detecting the level of a protein in a sample or a subject.

An “epitope” is an antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on NTHi PDE1.

As used herein “expression” refers to transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA.

In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression control sequences refer to nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adena-associated viruses) that incorporate the recombinant polynucleotide.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g., a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules. In one specific, non-limiting example, a heterologous nucleic acid molecule encoding a recombinant NTHi PDE1 or immunogenic fragment thereof, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

As used herein, “homologous proteins” are proteins that have a similar structure and function, for example, proteins from two or more species or viral strains that have similar structure and function in the two or more species or bacterial strains. Homologous proteins share similar protein folding characteristics and can be considered structural homologs.

Homologous proteins typically share a high degree of sequence conservation, such as at least 80%, at least 90%, at least 91%, at least 92%, at least 93%, at least 94%, or at least 95%, at least 96%, at least 97%, at least 98%, or at least 99% sequence conservation, and a high degree of sequence identity, such as at least 80%, at least 90%, at least 91%, at least 92%, at least 93%, at least 94%, or at least 95%, at least 96%, at least 97%, at least 98%, or at least 99% sequence identity.

By “host cell” is meant a cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Haemophilus influenzae is a small, non-motile, Gram-negative coccbacillus. “H. inflenzae infection and/or disease” refers to well-recognized constellation of signs and symptoms in persons infected by NTHi, as determined by antibody or western blot studies. NTHi causes a wide spectrum of human infections, including: asymptomatic colonization of the upper respiratory tract (i.e. carriage); infections that extend from colonized mucosal surfaces to cause otitis media (inflammation of the middle ear), bronchitis, conjunctivitis, sinusitis, urinary tract infections and pneumonia; and invasive infections, such as bacteremia, septic arthritis, epiglottitis, pneumonia, empyema, pericarditis, cellulitis, osteomyelitis and meningitis.

As used herein, “immune response” refers to a response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies. “Priming an immune response” refers to pre-treatment of a subject with an adjuvant to increase the desired immune response to a later administered immunogenic agent. “Enhancing an immune response” refers to co-administration of an adjuvant and an immunogenic agent, wherein the adjuvant increases the desired immune response to the immunogenic agent compared to administration of the immunogenic agent to the subject in the absence of the adjuvant.

As used herein, “immunogen” refers to a protein or a portion thereof that is capable of inducing an immune response in a mammal, such as a mammal infected or at risk of infection with a pathogen. An appropriate immunogenic preparation can contain, for example, a recombinantly expressed or a chemically synthesized polypeptide (e.g. synthesized by cell-free protein synthesis). Administration of an immunogen can lead to protective immunity and/or proactive immunity against a pathogen of interest. In some examples, an immunogen comprises a recombinant or synthesized NTHi PDE1 or immunogenic fragment thereof, as disclosed herein.

As used herein an “immunogenic composition” refers to a composition comprising an immunogenic polypeptide, or a nucleic acid molecule or vector encoding an immunogenic polypeptide that induces a measurable CTL response against the immunogenic polypeptide, or induces a measurable B cell response (such as production of antibodies) against the immunogenic polypeptide. In one example, an “immunogenic composition” is a composition that includes a disclosed recombinant or synthsized NTHi PDE1 or immunogenic fragment thereof, that induces a measurable CTL response against an H. influenzae, or induces a measurable B cell response (such as production of antibodies) against H. influenzae. It further refers to isolated nucleic acids encoding an antigen, such as a nucleic acid that can be used to express the antigen (and thus be used to elicit an immune response against this peptide). For in vitro use, an immunogenic composition may comprise or consist of the isolated protein or nucleic acid molecule encoding the protein. For in vivo use, the immunogenic composition will typically include the protein or nucleic acid molecule in a pharmaceutically acceptable carrier and may also include other agents, such as an adjuvant. Any particular protein, such as a disclosed recombinant or synthesized NTHi PDE1 or immunogenic fragment thereof, or a nucleic acid encoding the protein, can be readily tested for its ability to induce a CTL or B cell response by art-recognized assays. Immunogenic compositions can include adjuvants, which are well known to one of skill in the art.

As used herein, “immunogenic polypeptide” refers to a polypeptide which comprises an allele-specific motif, an epitope or other sequence such that the polypeptide will bind an MHC molecule and induce an immune response, such as a cytotoxic T lymphocyte (“CTL”) response, and/or a B cell response (for example, antibody production), and/or a T-helper lymphocyte response against the antigen from which the immunogenic polypeptide is derived.

An “isolated” biological component (such as a protein, for example a disclosed immunogen or nucleic acid encoding such an antigen) has been substantially separated or purified away from other biological components, such as other biological components in which the component naturally occurs, such as other chromosomal and extrachromosomal DNA, RNA, and proteins. Proteins, peptides and nucleic acids that have been “isolated” include proteins purified by standard purification methods. The term also embraces proteins or peptides prepared by recombinant expression in a host cell as well as chemically synthesized proteins, peptides and nucleic acid molecules. Isolated does not require absolute purity, and can include protein, peptide, or nucleic acid molecules that are at least 50% isolated, such as at least 75%, 80%, 90%, 95%, 98%, 99%, or even 99.9% isolated.

“K_{d}” is a dissociation constant for a given interaction, such as a polypeptide ligand interaction or an antibody antigen interaction. For example, for the bimolecular interaction of an antibody or antigen binding fragment and an immunogen (such as NTHi PDE1 polypeptide) it is the concentration of the individual components of the bimolecular interaction divided by the concentration of the complex.

The term “linker” refers to a bi-functional molecule that can be used to link two molecules into one contiguous molecule, for example, to link a carrier molecule to a immunogenic polypeptide. Non-limiting examples of peptide linkers include glycine-serine linkers, such as a (GGGGS)x linker or a 10 amino acid glycine-serine linker. Unless context indicates otherwise, reference to “linking” a first polypeptide and a second polypeptide (or to two polypeptides “linked” together) refers to covalent linkage by peptide bond, or (if a peptide linker is involved) covalent linkage of the first and second polypeptides to the N and C termini of a peptide linker. Typically, such linkage is accomplished using molecular biology techniques to genetically manipulate DNA encoding the first polypeptide linked to the second polypeptide by the peptide linker.

The terms “conjugating,” “joining,” “bonding,” can refer to making two molecules into one contiguous molecule; for example, joining two polypeptides into one contiguous polypeptide, or covalently attaching a carrier molecule or other molecule to an immunogenic polypeptide, such as an recombinant or synthesized NTHi PDE1 or immunogenic fragment thereof, as disclosed herein. The conjugate can be either by chemical or recombinant means. “Chemical means” refers to a reaction, for example, between the immunogenic polypeptide moiety and the carrier molecule such that there is a covalent bond formed between the two molecules to form one molecule.

A “neutralizing antibody” refers to an antibody which reduces the infectious titer of an infectious agent by binding to a specific antigen on the infectious agent. In some examples the infectious agent is a bacteria. In some examples, an antibody that is specific for NTHi PDE1 neutralizes the infectious titer of H. influenzae. A “broadly neutralizing antibody” is an antibody that binds to and inhibits the function of related antigens, such as antigens that share at least 65%, 75%, 85%, 90%, 95%, 96%, 97%, 98% or 99% identity antigenic surface of antigen. With regard to an antigen from a pathogen, such as bacterium, the antibody can bind to and inhibit the function of an antigen from more than one class and/or subclass of the pathogen.

As used herein, “nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, “operably linked” is a first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

As used herein, “pharmaceutically acceptable carriers” refer to pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired anti-NTHi immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

The term “polypeptide” refers to any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues. A protein can include multiple polypeptide chains. Additionally, a single contiguous polypeptide chain of amino acid residues can include multiple polypeptides.

“Polypeptide modifications” refers to polypeptides and peptides, such as the recombinant or synthesized NTHi PDE1 proteins disclosed herein can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide. Hydroxyl groups of the peptide side chains can be converted to C₁-C₁₆ alkoxy or to a C₁-C₁₆ ester using well-recognized techniques. Phenyl and phenolic rings of the peptide side chains can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability. For example, a C- or N-terminal cysteine can be added to the peptide, so that when oxidized the peptide will contain a disulfide bond, generating a cyclic peptide. Other peptide cyclizing methods include the formation of thioethers and carboxyl- and amino-terminal amides and esters.

“Prime-boost vaccination” is an immunotherapy including administration of a first immunogenic composition (the primer vaccine) followed by administration of a second immunogenic composition (the booster vaccine) to a subject to induce an immune response. The primer vaccine and/or the booster vaccine include a vector (such as a viral vector, RNA, or DNA vector) expressing the antigen to which the immune response is directed. The booster vaccine is administered to the subject after the primer vaccine; the skilled artisan will understand a suitable time interval between administration of the primer vaccine and the booster vaccine. In some embodiments, the primer vaccine, the booster vaccine, or both primer vaccine and the booster vaccine additionally include an adjuvant.

“Protein nanoparticle” refers to a multi-subunit, protein-based polyhedron shaped structure. The subunits are each composed of proteins or polypeptides (for example a glycosylated polypeptide), and, optionally of single or multiple features of the following: nucleic acids, prosthetic groups, organic and inorganic compounds. Non-limiting examples of protein nanoparticles include gold or ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct. and Mol. Biol., 15:939-947, 2008, incorporated by reference herein), Sulfur Oxygenase Reductase (SOR) nanoparticles (see, e.g., Urich et al., Science, 311:996-1 000, 2006, incorporated by reference herein), lumazine synthase nanoparticle (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Gold, ferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. In some examples, gold, ferritin, encapsulin, SOR, lumazine synthase, or pyruvate dehydrogenase monomers are linked to a recombinant NTHi PDE1 or immunogenic fragment thereof and self-assemble into a protein nanoparticle presenting the recombinant NTHi immunogenic peptide or fragment on its surface, which can be administered to a subject to stimulate an immune response to the antigen

As used herein, “recombinant” refers to a nucleic acid that has a sequence that is not naturally occurring or has a sequence that is made by an artificial combination of two otherwise separated segments of sequence. This artificial combination can be accomplished, for example, the artificial manipulation of isolated segments of nucleic acids, for example using genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

A “sample (or biological sample) is a biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material.

As used herein, “sequence identity” refers to the similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CAB/OS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166/1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

Homologs and variants of a polypeptide are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Sambrook et al. (Molecular Cloning: A Laboratory Manual, 4^{th} ed, Cold Spring Harbor, N.Y., 2012) and Ausubel et al. (In Current Protocols in Molecular Biology, John Wiley & Sons, New York, through supplement 104, 2013). One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CAB/OS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989). An oligonucleotide is a linear polynucleotide sequence of up to about 100 nucleotide bases in length.

As used herein, reference to “at least 80% identity” (or similar language) refers to “at least 80%, at least 85%, at least 90%, at least 91%, at least 92%, at least 93%, at least 94%, at least 95%, at least 96%, at least 97%, at least 98%, at least 99%, or even 100% identity” to a specified reference sequence. As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

The term “specifically bind” is referring to the formation of an antibody:antigen protein complex, or a protein:protein complex, refers to a binding reaction which determines the presence of a target protein, peptide, or polysaccharide (for example a glycoprotein), in the presence of a heterogeneous population of proteins and other biologics. Thus, under designated conditions, an particular antibody or protein binds preferentially to a particular target protein, peptide or polysaccharide (such as an antigen present on the surface of a pathogen, for example NTHi PDE1) and does not bind in a significant amount to other proteins or polysaccharides present in the sample or subject. Specific binding can be determined by methods known in the art. A first protein or antibody specifically binds to a target protein when the interaction has a K_{D} of less than 10⁻⁶ Molar, such as less than 10⁻⁷ Molar, less than 10⁻⁸ Molar, less than 10⁻⁹, or even less than 10⁻¹⁰ Molar.

As used herein, “subject” refers to a living multi-cellular vertebrate organisms, a category that includes human and non-human mammals. In an example, a subject is a human. In an example, a subject is selected that is in need of inhibiting of an H. influenzae infection. For example, the subject is either uninfected and at risk of H. influenzae infection or is infected in need of treatment.

“T Cell” is a white blood cell critical to the immune response. T cells include, but are not limited to, CD4⁺ T cells and CD8⁺ T cells. A CD4⁺ T lymphocyte is an immune cell that expresses CD4 on its surface. These cells, also known as helper T cells, help orchestrate the immune response, including antibody responses as well as killer T cell responses. Th1 and Th2 cells are functional subsets of helper T cells. Th1 cells secrete a set of cytokines, including interferon-gamma, and whose principal function is to stimulate phagocyte-mediated defense against infections, especially related to intracellular microbes. Th2 cells secrete a set of cytokines, including interleukin (IL)-4 and IL-5, and whose principal functions are to stimulate IgE and eosinophil/mast cell-mediated immune reactions and to downregulate Th1 responses.

As used herein, “therapeutically effective amount” refers to the amount of agent, such as a disclosed immunogen or immunogenic composition that is sufficient to prevent, treat (including prophylaxis), reduce and/or ameliorate the symptoms and/or underlying causes of a disorder or disease, for example to prevent, inhibit, and/or treat H. influenzae infection. In some embodiments, a therapeutically effective amount is sufficient to reduce or eliminate a symptom of a disease, such as H. influenzae infection. For instance, this can be the amount necessary to inhibit or prevent replication or to measurably alter outward symptoms of the infection. In general, this amount will be sufficient to measurably inhibit H. influenzae replication or infectivity.

In one example, a desired response is to inhibit or reduce or prevent H. influenzae infection. The H. influenzae from the infected tissues do not need to be completely eliminated or reduced or prevented for the composition to be effective. For example, administration of a therapeutically effective amount of the agent can decrease the number of H. influenzae (or prevent the infection of tissues) by a desired amount, for example by at least 50%, at least 60%, at least 70%, at least 80%, at least 90%, at least 95%, at least 98%, or even at least 100% (elimination or prevention of detectable H. influenzae), as compared to the number of H. influenzae infected tissues in the absence of the composition.

It is understood that to obtain a protective immune response against a pathogen can require multiple administrations of the immunogenic composition. Thus, a therapeutically effective amount encompasses a fractional dose that contributes in combination with previous or subsequent administrations to attaining a protective immune response. For example, a therapeutically effective amount of an agent can be administered in a single dose, or in several doses, for example daily, during a course of treatment (such as a prime-boost vaccination treatment).

However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

“Treating or preventing a disease” refers to inhibiting the full development of a disease or condition, for example, in a subject who is at risk for a H. influenzae disease such as otitis media. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the bacterial load, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

The term “reduces” is a relative term, such that an agent reduces a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, an immunogenic composition that reduces or prevents an infection or a response, can, but does not necessarily completely, eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

As used herein, “vaccine” refers to a pharmaceutical composition that elicits a prophylactic or therapeutic immune response in a subject. In some cases, the immune response is a protective immune response. Typically, a vaccine elicits an antigen-specific immune response to an antigen of a pathogen, for example a bacterial pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine reduces the severity of the symptoms associated with H. influenzae infection and/or decreases the bacterial load compared to a control. In another non-limiting example, a vaccine reduces H. influenzae infection compared to a control.

The term “vector” refers to a nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function. For example, such that the viral vector does not replicate in typical host cells, especially those in a human patient that could be infected by the viral vector in the course of a therapeutic method.

“Virus-like particle” (VLP) is a non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354: 53073, 2012).

### PED1 Immunogens

In one aspect, the present invention involves a PDE1 peptide, or a portion thereof, that is an effective immunogen.

In a further aspect, the invention includes a truncated PDE1. In NTHi a self-cleavage site at Asn269 can destroy the thiamine pyrophosphate (TPP) binding domain and expose a potential GG-motif leader sequence that may direct export through an ATP-binding cassette (ABC) transporter. The truncated N-terminal TPP binding domain of PDE1 could fold into a significantly different structure that is no longer capable of enzymatic activity. Without wishing to be bound by theory, it is believed that under low nutrient conditions, dissociation of TPP from PDE1 induces self-cleavage to generate a new functional protein. It is expected that the moonlighting functions of PDE1 play a significant role in the invasive pathogenesis of NTHi that follows respiratory viral infection.

The invention provides polypeptides comprising the H. infulenzae amino acid sequences disclosed herein. Further, the invention provides polypeptides comprising amino acid sequences that have sequence identity to the H. influenza amino acid sequences disclosed herein. Depending on the particular sequence, the degree of sequnces identity is preferably greater than 50% (e.g. 60%, 70%, 80%, 90%, 91%, 92%, 93%, 94%, 95%, 96%, 97%, 98%, 99%, or greater). These polypeptides include homologs, orthologs, allelic avriants and functional mutants. Typically, 50% identity or more between two polypeptide sequences is considered to be an indication of functional equivalence. Identity between polypeptides is preferably determined by the Smith-Waterman homology search alogorithm as implemented in the MPSRCH program (Oxford Molecular), using a affine gap search with parameters gap open penalty+12 and gap extension penalty=1.

These polypeptide may include one or more (e.g. 1, 2, 3, 4, 5, 6, 7, 8, 9, 10, etc.) conservative amino acid replacements i.e. replacements of one amino acid with another which has a related side chain. The polypeptides may also include one or more (e.g. 1, 2, 3, 4, 5, 6, 7, 8, 9, 10, etc.) single amino acid deletions relative to the NTHi sequences of the examples. The polypeptides may also include one or more (e.g. 1, 2, 3, 4, 5, 6, 7, 8, 9, 10, etc.) insertions (e.g. each of 1, 2, 3, 4 or 5 amino acids) relative to the NTHi sequences of the examples.

The invention further provides polypeptides comprising fragments of the H. influenzae amino acid sequences disclosed in the examples. The fragments should comprise at least n consecutive amino acids from the sequences and, depending on the particular sequence, n is 7 or more (e.g. 8, 10, 12, 14, 16, 18, 20, 22, 24, 26, 28, 30, 35, 40, 45, 50, 60, 70, 80, 90, 100 or more).

The fragment may comprise at least one T-cell or, preferably, a B-cell epitope of the sequence. T- and B-cell epitopes can be identified empirically (e.g. using PEPSCAN or similar methods), or they can be predicted (e.g. using the Jameson-Wolf antigenic index, matrix-based approaches, TEPITOPE, neural networks, OptiMer & EpiMer, ADEPT, Tsites, hydrophilicity, or antigenic index).

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis. Solid-phase peptide synthesis is preferred, such as methods based on tBoc or Fmoc chemistry. Enzymatic synthesis may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non-natural amino acids, such as iodotyrosine or methylphenylalanine, azidohomoalanine, etc.). Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

### Antibodies

The invention provides antibodies that bind to polypeptides of the invention. These may be polyclonal or monoclonal and may be produced by any suitable means (e.g. by recombinant expression). To increase compatibility with the human immune system, the antibodies may be chimeric or humanised, or fully human antibodies may be used. The antibodies may include a detectable label (e.g. for diagnostic assays). Antibodies of the invention may be attached to a solid support. Antibodies of the invention are preferably neutralizing antibodies.

Monoclonal antibodies are particularly useful in identification and purification of the individual polypeptides against which they are directed. Monoclonal antibodies of the invention may also be employed as reagents in immunoassays, radioimmunoassays (RIA) or enzyme-linked immunosorbent assays (ELISA), etc. In these applications, the antibodies can be labelled with an analytically-detectable reagent such as a radioisotope, a fluorescent molecule or an enzyme. The monoclonal antibodies produced by the above method may also be used for the molecular identification and characterization (epitope mapping) of polypeptides of the invention. Antibodies of the invention are preferably provided in purified or substantially purified form. Typically, the antibody will be present in a composition that is substantially free of other polypeptides e.g. where less than 90% (by weight), usually less than 60% and more usually less than 50% of the composition is made up of other polypeptides.

The invention provides a process for detecting polypeptides of the invention, comprising the steps of: (a) contacting an antibody of the invention with a biological sample under conditions suitable for the formation of an antibody-antigen complexes; and (b) detecting said complexes.

The invention provides a process for detecting antibodies of the invention, comprising the steps of: (a) contacting a polypeptide of the invention with a biological sample (e.g. a blood or serum sample) under conditions suitable for the formation of an antibody-antigen complexes; and (b) detecting said complexes.

### Nucleic Acids

The invention provides nucleic acid comprising the H. influenzae nucleotide sequences as disclosed herein. The invention also provides nucleic acid comprising nucleotide sequences having sequence identity to the H. influenzae nucleotide sequences disclosed herein or fragments thereof. The invention also provides nucleic acid which can hybridize to the H. influenzae nucleic acid disclosed in the examples. Hybridization reactions can be performed under conditions of different “stringency”. Conditions that increase stringency of a hybridization reaction of widely known. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art.

Nucleic acid comprising fragments of these sequences are also provided. These should comprise at least n consecutive nucleotides from the H. influenzae sequences and, depending on the particular sequence n is 10 or more (e.g. 12, 14, 15, 18, 20, 25, 30, 35, 40, 50, 60, 70, 80, 90, 100, 150, 200 or more).

The invention also provides nucleic acid encoding the polypeptides and polypeptide fragments of the invention.

The invention includes nucleic acid comprising sequences complementary to the sequences disclosed in the sequence listing (e.g. for antisense or probing, or for use as primers), as well as the sequences in the orientation actually shown. Nucleic acids of the invention can be used in hybridization reactions (e.g. Northern or Southern blots, or in nucleic acid microarrays or ‘gene chips’) and amplification reactions (e.g. PCR, SDA, SSSR, LCR, TMA, NASBA, etc.) and other nucleic acid techniques.

Nucleic acid according to the invention can take various forms (e.g. single-stranded, double-stranded, vectors, primers, probes, labelled etc.). Nucleic acids of the invention maybe circular or branched, but will generally be linear. Unless otherwise specified or required, any embodiment of the invention that utilizes a nucleic acid may utilize both the double-stranded form and each of two complementary single stranded forms which make up the double-stranded form. Primers and probes are generally single-stranded, as are antisense nucleic acids. The invention provides vectors comprising the nucleotide sequences of the invention (e.g. cloning or expression vectors) and host cells transformed with such vectors.

### EXAMPLES

### Example 1: PED1 as a Moonlighting Protein

There is ample evidence of many bacterial proteins moonlighting with additional functions. Specific examples include GAPDH serving as a transferrin binding protein in Staphylococcus or binding to uPAR-CD87 receptor and thus acting as an adhesion in S. pyogenes. In Haemophilus, it appears that there is only one protein, derived from aspA, which moonlights and binds to plasminogen. The role of PDE1 as a moonlighting protein has not been described in NTHi, and has important implications as a virulence factor, adhesion molecule, and/or in helping to utilize alternative energy sources. Examination of this key protein with respect to these alternative properties is novel and highlight important findings in the pathogenesis of this pathogen. There are no other studies that have found a truncated PDE1 in gram-negative bacteria and thus this phenomenon could be unique to NTHi.

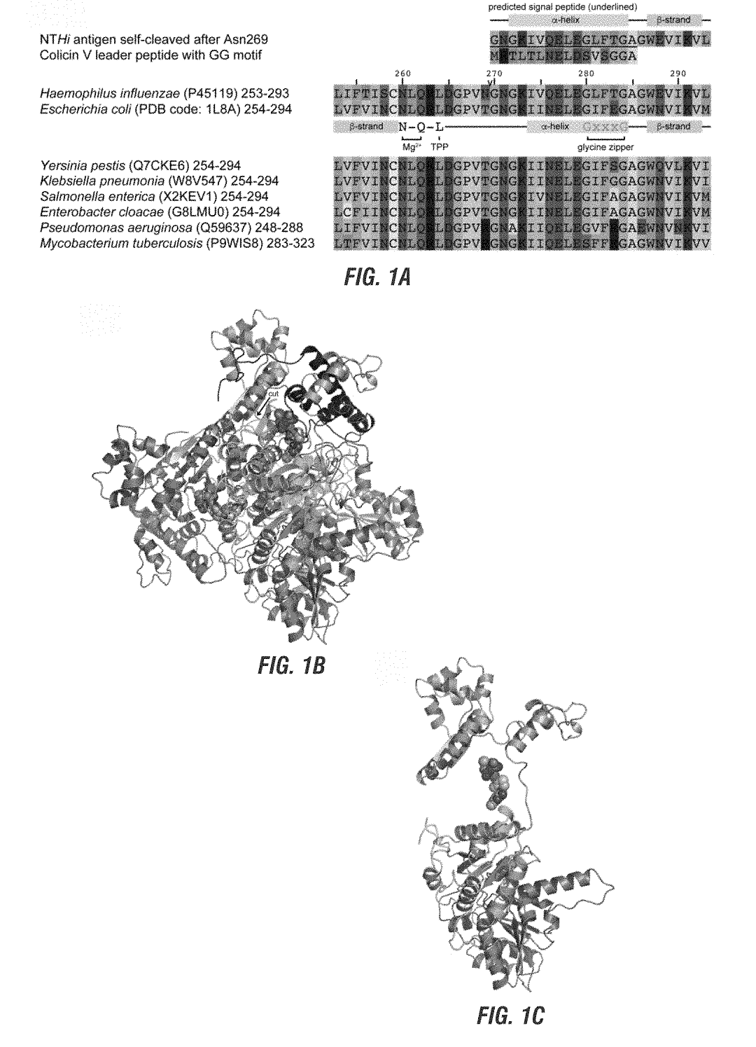

No prior studies are known to have shown how a truncated PDE1 could function or how it would move through the cytoplasm. Thus structural analysis of the protein is key to understanding this. The Inventors have preliminarily screened PDE1's known structure (FIG. 1) and found evidence that suggests PDE1 has a unique cleavage site at Asn269 that might expose a transport signal peptide after cleavage. Protein modeling and x-ray crystallography provides a better understand of the truncated 69 kDa portion of PDE1 complex. Of interest, a truncated PDE1 monomer would be forced to undergo significant refolding possibly changing its behavior (function or interaction with other proteins). Of note, the 30 kDa protein as it appears to be degraded in some but not all preliminary experiments.

### Example 2: PDE1 as an Immunogen

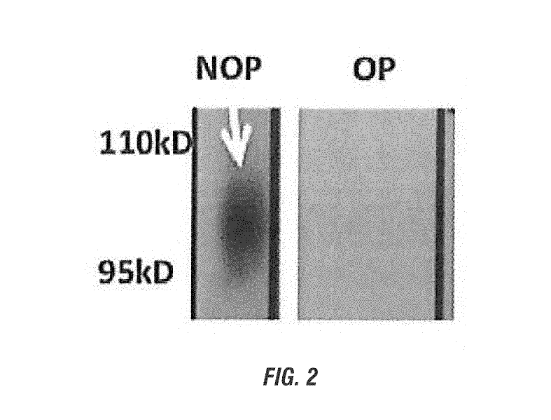

The discovery of truncated PDE1 came from examination of the serum responses from 50 children who are either otitis-prone or non-prone (25 in each group). Of interest, all of the non-prone children mounted an antibody response to PDE1 while none of the otitis-prone children mounted antibodies to linear epitopes (FIG. 2) within PDE1. These data suggest that PDE1 is a significant candidate for a NTHi vaccine. A better understanding of the mechanism and function of PDE1 in NTHi pathogenesis helps in designing strategies to target it through vaccination or therapeutics. While multiple proteins have been tested in mice as vaccine candidates, none have succeeded in clinical trials (8% efficacy using protein D). Thus, additional protein immunogens are necessary to design efficacious vaccines to prevent NTHi invasive diseases.

The truncation of PDE1 improves the possibility of using this as an immunogen for an NTHi vaccine since there should be unique conformational epitopes in the refolded N-terminus after cleavage. Also, there are significant differences in sequence between the homodimeric PDE1 and heterotetrameric PDE1 alpha and beta genes (22). Thus, the PDE1 immunogen may be a bad vaccine strategy for M. pneumoniae due to the similarity in PDE1 alpha with the human mitochondrial isoform, but it may work for NTHi as it appears sufficiently different to avoid autoimmunity issues.

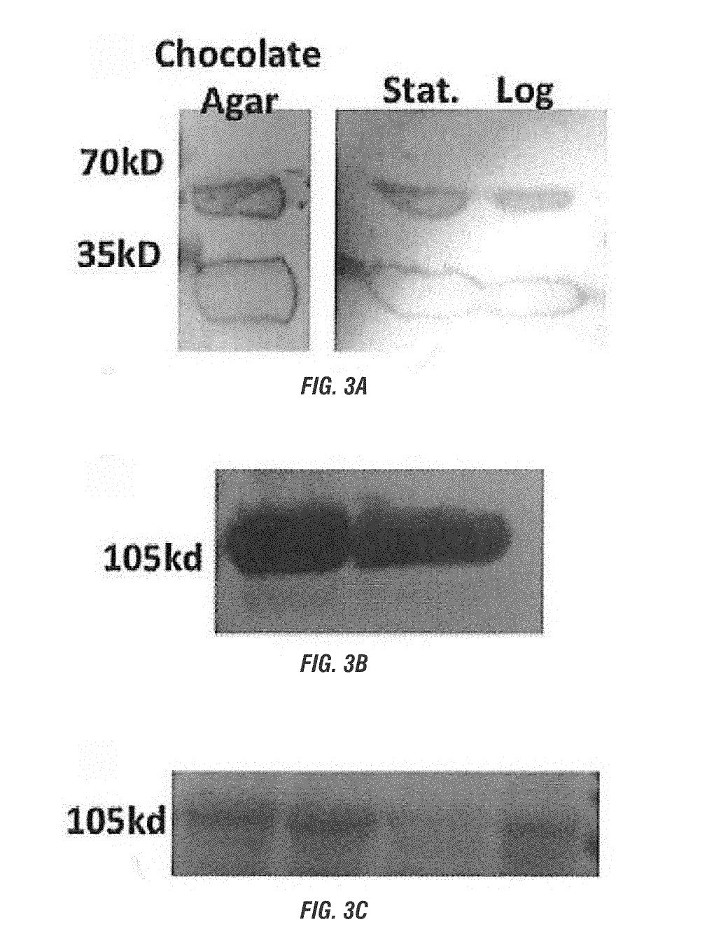

NTHi chocolate Agar plates were grown and the cells used in a western blotting for PDE1 (FIG. 3A). A significant band occurred at the 69 kDa and 30 kDa sizes. Also, the bacteria was grown in log and stationary phases and similar full-length and cleaved PDE1 proteins were observed. These experiments were conducted using a cross-reactive mammalian PDE1 antibody. We also detected full length protein in the agar and stationary growth (not shown). We next cloned the aceE (PDE1) gene from NTHi and expressed it in E. coli using T7 expression plasmid. We found a large band at 105 kDa representing the full size (with 6×His) of PDE1 in E. coli with no truncation (FIG. 3B).

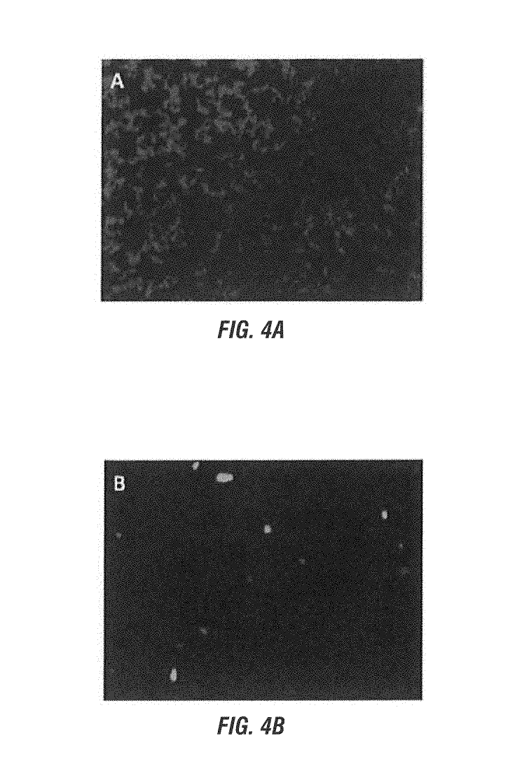

NTHi was grown under permissive culture conditions to log and stationary phase. To a fraction of cells, we disrupted the surface membrane by brief incubation in low salt buffer and stained the cells with anti-PDE1 antibody (FIG. 4). The other cells underwent no membrane disruption. Of interest, we found light staining of cells stained for outer membrane expression in the log or stationary growth stages. We found significant staining of cells that we disrupted the membrane in the log phase but not the stationary phase. These data suggest that PDE1 may be localizing into the periplasm during growth.

Wildtype PDE1 from H. influenzae
(SEQ ID NO: 1)
MSEILKNDVDPIETQDWLQSLDSLIREEGVERAQYIVEQVIGQARTSGVS
LPTGVTTDYVNTIPVAEQPAYPGDHAIERRIRSAVRWNAIAMVLRSQKKD
LDLGGHISTFQSAATMYEVCYNHFFKAATEKNGGDLIFFQGHAAPGMYAR
AFLEGRLTEEQMDNFRQEAFTDGLSSYPHPKLMPEFWQFSTVSMGLGPVN
AIYQARFLKYLDNRGLKDTKDQKVYAFLGDGEMDEIESKGALTFAAREHL
DNLIFTISCNLQRLDGPVNGNGKIVQELEGLFTGAGWEVIKVLWGSDWDK
LFAKDTSGKLTQLMMEVVDGDYLTFKSKDGAYIREHFFGRYPETAALVAD
MTDDEIWALRRGAHDSEKLYAAYAKAQNATKPVVILAHQVKGYKIPEAES
KNTAHQSKKMSYESLKGFRDFFELPLTDEQVEKLEYIKFAEGTPEYEYLH
GHRKALNGYVPARRTKFDVEYKVPALEEFKALLEEQPRGISTTMAFTRAL
NILLKDKNIGKTIVPMIADEARTFGMEGLFRQVGIYNPHGQNYIPSDRDL
VAYYREAKDGQVLQEGINELGATASWLAAANSYSVNNQPMIPFFIYYSMF
GFQRVGDMMWAAGDQLARGFMVGGTSGRTTLNGEGLQHEDGHSHIQAGII
PNCITYDPSFAFEVAVIMQDGINRMYGEKQEDVFYYMTTLNEVMDQPAMP
AGAEEGIRKGLYKFETVEGKKGKGHVQLLGSGAIMRHVREAAQILANDYG
VTSDVFSAPSFNELAREGHDAARWNLLHPTETQRVPYVAQVLADLPTVAS
TDYVKGYADQIRAFVPSKHYHVLGTDGFGRSDSRANLREHFEVDARYVVV
AALSQLAKEGTVSNQVVADAIAKFGLNVDRINPLYA
Immunogenic PDE1 from H. influenzae
(SEQ ID NO: 2)
GNGKIVQELEGLFTGAGWEVIKVLWGSDWDKLFAKDTSGKLTQLMMEVVD
GDYLTFKSKDGAYIREHFFGRYPETAALVADMTDDEIWALRRGAHDSEKL
YAAYAKAQNATKPVVILAHQVKGYKIPEAESKNTAHQSKKMSYESLKGFR
DFFELPLTDEQVEKLEYIKFAEGTPEYEYLHGHRKALNGYVPARRTKFDV
EYKVPALEEFKALLEEQPRGISTTMAFTRALNILLKDKNIGKTIVPMIAD
EARTFGMEGLFRQVGIYNPHGQNYIPSDRDLVAYYREAKDGQVLQEGINE
LGATASWLAAANSYSVNNQPMIPFFIYYSMFGFQRVGDMMWAAGDQLARG
FMVGGTSGRTTLNGEGLQHEDGHSHIQAGIIPNCITYDPSFAFEVAVIMQ
DGINRMYGEKQEDVFYYMTTLNEVMDQPAMPAGAEEGIRKGLYKFETVEG
KKGKGHVQLLGSGAIMRHVREAAQILANDYGVTSDVFSAPSFNELAREGH
DAARWNLLHPTETQRVPYVAQVLADLPTVASTDYVKGYADQIRAFVPSKH
YHVLGTDGFGRSDSRANLREHFEVDARYVVVAALSQLAKEGTVSNQVVAD
AIAKFGLNVDRINPLYA

What is claimed is:

1. An isolated immunogen, comprising,
a recombinant NTHi PDE1 or immunogenic fragment/truncation thereof

2. The immunogen of claim 1, wherein said NTHi PDE1 is truncated at Asn269 (SEQ ID NO:2)

3. The immunogen of claim 1, comprising an amino acid sequence at least 50% identical to NTHi PDE1 or immunogenic fragment thereof.

4. A protein nanoparticle comprising the recombinant NTHi PDE1 or immunogenic fragment thereof of claim 1.

5. The protein nanoparticle of claim 4, wherein the protein nanoparticle is a gold nanoparticle, a ferritin nanoparticle, an encapulin nanoparticle, a Sulfur Oxygenase reductase (SOR) nanoparticle, a lumazine nanoparticle or pyruvate dehydrogenase nanoparticle.

6. The protein nanoparticle of claim 5, wherein the protein nanoparticle comprises two or more of the recombinant NTHi PDE1 or immunogenic fragments thereof.

7. An isolated nucleic acid molecule encoding the recombinant NTHi PDE1 or immunogenic fragment thereof.

8. The nucleic acid of claim 7, wherein the nucleic acid molecule encodes NTHi PDE1 or a polypeptide at least 80% identical thereto.

9. The nucleic acid of claim 8, operably linked to a promoter.

10. A vector comprising the nucleic acid molecule of claim 9.

11. An isolated host cell comprising the vector of claim 10.

12. A protein nanoparticle, virus-like particle, nucleic acid molecule, or vector comprising the immunogen of any one of the prior claims, wherein administration of an effective amount of the immunogen, protein nanoparticle, nucleic acid molecule, or vector induces a neutralizing response to H. influenzae in the subject.

13. An immunogenic composition comprising an effective amount of the immunogen, protein nanoparticle, nucleic acid molecule, or vector of any one of the prior claims, and a pharmaceutically acceptable carrier.

14. The immunogenic composition of claim 13, further comprising an adjuvant.

15. A method for generating an immune response to H. influenzae in a subject, comprising administering to the subject an effective amount of the immunogenic composition of claim 13, thereby generating the immune response.

16. A method for preventing or treating a H. influenzae infection in a subject, comprising administering to the subject a therapeutically effective amount of the immunogenic composition of claim 13, thereby treating the subject or preventing H. influenzae infection of the subject.

17. The method of claim 15, comprising a prime-boost administration of the immunogenic composition.

18. A method for detecting or isolating an antibody that specifically binds to H. influenza PDE1 or fragment thereof from a subject, comprising:
providing an effective amount of the immunogen of any one of claim 1;
contacting a biological sample from the subject with the immunogen under conditions sufficient to form an immune complex between the immunogen and the antibody that specifically binds to H. influenza PDE1 or fragment thereof; and detecting the immune complex, thereby detecting or isolating the antibody from the subject.

19. The method of claim 15, wherein the subject is at risk of or has an H. influenza infection.

20. A kit comprising the immunogen, protein nanoparticle, nucleic acid molecule, vector, or composition of claim 1, and instructions for using the kit.


SEQUENCE LISTING (12 sequences)

SEQ ID NO: 1 - protein, 886 aa, Haemophilus influenzae
MSEILKNDVDPIETQDWLQSLDSLIREEGVERAQYIVEQVIGQARTSGVSLPTGVTTDYVNTIPVAEQPAYPGDHAIERRIRSAVRWNAIAMVLRSQKKDLDLGGHISTFQSAATMYEVCYNHFFKAATEKNGGDLIFFQGHAAPGMYARAFLEGRLTEEQMDNFRQEAFTDGLSSYPHPKLMPEFWQFSTVSMGLGPVNAIYQARFLKYLDNRGLKDTKDQKVYAFLGDGEMDEIESKGALTFAAREHLDNLIFTISCNLQRLDGPVNGNGKIVQELEGLFTGAGWEVIKVLWGSDWDKLFAKDTSGKLTQLMMEVVDGDYLTFKSKDGAYIREHFFGRYPETAALVADMTDDEIWALRRGAHDSEKLYAAYAKAQNATKPVVILAHQVKGYKIPEAESKNTAHQSKKMSYESLKGFRDFFELPLTDEQVEKLEYIKFAEGTPEYEYLHGHRKALNGYVPARRTKFDVEYKVPALEEFKALLEEQPRGISTTMAFTRALNILLKDKNIGKTIVPMIADEARTFGMEGLFRQVGIYNPHGQNYIPSDRDLVAYYREAKDGQVLQEGINELGATASWLAAANSYSVNNQPMIPFFIYYSMFGFQRVGDMMWAAGDQLARGFMVGGTSGRTTLNGEGLQHEDGHSHIQAGIIPNCITYDPSFAFEVAVIMQDGINRMYGEKQEDVFYYMTTLNEVMDQPAMPAGAEEGIRKGLYKFETVEGKKGKGHVQLLGSGAIMRHVREAAQILANDYGVTSDVFSAPSFNELAREGHDAARWNLLHPTETQRVPYVAQVLADLPTVASTDYVKGYADQIRAFVPSKHYHVLGTDGFGRSDSRANLREHFEVDARYVVVAALSQLAKEGTVSNQVVADAIAKFGLNVDRINPLYA

SEQ ID NO: 2 - protein, 617 aa, Haemophilus influenzae
GNGKIVQELEGLFTGAGWEVIKVLWGSDWDKLFAKDTSGKLTQLMMEVVDGDYLTFKSKDGAYIREHFFGRYPETAALVADMTDDEIWALRRGAHDSEKLYAAYAKAQNATKPVVILAHQVKGYKIPEAESKNTAHQSKKMSYESLKGFRDFFELPLTDEQVEKLEYIKFAEGTPEYEYLHGHRKALNGYVPARRTKFDVEYKVPALEEFKALLEEQPRGISTTMAFTRALNILLKDKNIGKTIVPMIADEARTFGMEGLFRQVGIYNPHGQNYIPSDRDLVAYYREAKDGQVLQEGINELGATASWLAAANSYSVNNQPMIPFFIYYSMFGFQRVGDMMWAAGDQLARGFMVGGTSGRTTLNGEGLQHEDGHSHIQAGIIPNCITYDPSFAFEVAVIMQDGINRMYGEKQEDVFYYMTTLNEVMDQPAMPAGAEEGIRKGLYKFETVEGKKGKGHVQLLGSGAIMRHVREAAQILANDYGVTSDVFSAPSFNELAREGHDAARWNLLHPTETQRVPYVAQVLADLPTVASTDYVKGYADQIRAFVPSKHYHVLGTDGFGRSDSRANLREHFEVDARYVVVAALSQLAKEGTVSNQVVADAIAKFGLNVDRINPLYA

SEQ ID NO: 3 - protein, 24 aa, Haemophilus influenzae
GNGKIVQELEGLFTGAGWEVIKVL

SEQ ID NO: 4 - protein, 16 aa, Escherichia coli
MRTLTLNELDSVSGGA

SEQ ID NO: 5 - protein, 41 aa, Haemophilus influenzae
LIFTISCNLQRLDGPVNGNGKIVQELEGLFTGAGWEVIKVL

SEQ ID NO: 6 - protein, 41 aa, Escherichia coli
LVFVINCNLQRLDGPVTGNGKIINELEGIFEGAGWNVIKVM

SEQ ID NO: 7 - protein, 41 aa, Yersinia pestis
LVFVINCNLQRLDGPVTGNGKIINELEGIFSGAGWQVLKVI

SEQ ID NO: 8 - protein, 41 aa, Klebsiella pneumoniae
LVFVINCNLQRLDGPVTGNGKIINELEGIFGGAGWNVIKVI

SEQ ID NO: 9 - protein, 41 aa, Salmonella enterica
LVFVINCNLQRLDGPVTGNGKIVNELEGIFAGAGWNVIKVM

SEQ ID NO: 10 - protein, 41 aa, Enterobacter cloacae
LCFIINCNLQRLDGPVTGNGKIINELEGIFAGAGWNVIKVM

SEQ ID NO: 11 - protein, 41 aa, Pseudomonas aeruginosa
LIFVINCNLQRLDGPVRGNAKIIQELEGVFRGAEWNVNKVI

SEQ ID NO: 12 - protein, 41 aa, Mycobacterium tuberculosis
LTFVINCNLQRLDGPVRGNGKIIQELESFFRGAGWNVIKVV